(19) United States
(12) Patent Application Publication
(10) Pub. No.: US 2006/0189869 A1
(43) Pub. Date: Aug. 24, 2006

(54) Systems and methods of 3 dimensional ultrasonic imaging of hard tissue

(76) Inventors: Natan Sela, St Rehovot (IL); Shmuel Bushpan, St Rehovot (IL); Lior Cohen, St Rehovot (IL); Michael Kardosh, St Rehovot (IL)

Correspondence Address: Mark M Friedman;Bill Polkinghorn, Discovery Dispatch, 9003 Florin Way, Upper Marlboro, MD, 20772, US

(21) Appl. No.: 10/500,800

(22) PCT Filed:
(86) PCT No.: PCT/IL03/00015

Related U.S. Application Data
(60) Provisional application No. 60/344,803, filed on Jan. 7, 2002.
(60) Provisional application No. 60/361,091, filed on Mar. 1, 2002.

(51) Int. Cl. A61B 8/00; A61M 16/00

(52) U.S. Cl. 600/443; 128/916

(57) ABSTRACT

A system (60) and method (20) of creating an ultrasonic image (48) of a hard tissue (70) within a target (68). The method (20) includes comprising transmitting (22) from a defined location (64) a beam energy (66) towards the target (68), adjusting (24) the angle of incidence between the beam (66) the hard tissue (70) to a normal angle, receiving (26) an echo-reflection at said defined location (64), calculating (32) a set of position co-ordinates (46) the surface causing the echo-reflection repeating (36) the sequence from additional locations (64), compiling (38) position co-ordinates (46) to generate a map (48) of the surface (69) causing the echo-reflection (65), and determining (40) a map (48) which represents a surface (69) of the hard tissue (70) by a predetermined rule. An additional method (90) employing reception of the echo reflection (60) at additional defined locations (64) is disclosed, as are systems (60) for performance of the claimed methods (20) (90).

This application claims priority from U.S. Patent application 60/344,803 filed on Jan. 7, 2002 and from U.S. Patent application 60/361,091 filed on Mar. 1, 2002

### FIELD AND BACKGROUND OF THE INVENTION

The present invention relates to systems and methods of 3 dimensional ultrasonic imaging of hard tissue and, more particularly, to a system and method of imaging imperfections in bone (e.g. fractures, jount abnormalities and implanted surgical anchors.) Preferably, images produced by the system additionally contain depictions of soft tissue surrounding the bone, as part of an integrated 3D rendering.

Orthopaedic medicine has traditionally relied upon radio-graphic images (e.g. X-Ray or CT scan) of bone tissue as a means of diagnosing bone abnormalities including fractures and malformations. These methods require exposing a patient to radiation. Significant amounts of radiation are required in order to produce 3D images using these techniques. Further, the equpment required for the practice of these techniques is typically expensive and of limited portability. Ultrasound offers a promising alternative to these prior art methods of bone imaging as will be discussed below.

The concept of 3 dimensional ultrasonic imaging is not unknown. However, it has traditionally been applied to soft tissue as exemplified in this first group of prior art references.

U.S. Pat. No. 4,100,916 issued to King describes an apparatus for collecting three-dimensional data pertaining to animal soft tissue organ structures. Teachings of King are strictly limited to soft tissue imaging and contain neither a hint nor suggestion that renderings of bone or imperfections therein may be produced by ultrasound.

U.S. Pat. No. 4,798,210 issued to Ledley describes a method for developing a 3D image of a 3D object using ultrasound whereby a first image is combined with a second image in order to create a 3D image. Again, teachings of Ledley contain neither a hint nor suggestion that renderings of bone or imperfections therein may be produced by ultrasound.

U.S. Pat. No. 5,924,989 issued to Polz is an additional example of a three dimensional ultrasonic system for capturing images of dynamic organs such as the heart or other parts of the respiratory system. Like Ledley. Polz employs a combination of different images in order to complete the three dimensional image. Again, teachings of Polz contain neither a hint nor suggestion that renderings of bone or imperfections therein may be produced by ultrasound.

U.S. Pat. No. 5,928,151 issued to Hossack et al. is a further example of a three dimensional ultrasonic scanning system. Again, teachings of this patent contain neither a hint nor suggestion that renderings of bone or imperfections therein may be produced by ultrasound. The opposite is true, the emphasis on the ability to work without contrast agents suggests that Hossack envisioned only soft tissue applications.

U.S. Pat. No. 6,120,453 issued to Sharp is a three dimensional ultrasound system. The teachings of this patent are similar to those of Ledley and Polz. Again, Sharp employs the combination of several images to create a three dimensional image. Again, Sharp offers neither a hint nor suggestion that renderings of bone or imperfections therein may be produced by ultrasound.

In summary, none of the patents in this first group even imply that generation of 3D images of bone can be generated using ultrasound technology. Instead, they stress various means of increasing resolution of 3D images of soft tissue. Application of these methods directly to hard tissue is impractical because the echo reflection properties of soft tissue are not similar to those of hard tissue.

The concept of ultrasonic imaging of bone is also not unknown. However, bone images produced by ultrasound are typically not three dimensional as exemplified by this second group of prior art references.

U.S. Pat. No. 4,476,873 issued to Sorenson et al. is an ultrasound scanning system used for imaging skeletal structure. This scanning system can distinguish between hard and soft tissue and is used to detect scoliosis. However, FIGS. 14-18 of this patent make it abundantly clear that while data may be collected in three dimensions, output is supplied as graphs. Thus, it is an inherent disadvantage of Sorenson that images are not provided as a result of the scan. It follows that three dimensional images of bone are not provided. Further, Sorenson teaches differentiation between lungs containing air and bones. It will be appreciated that lung tissue, which presents alternating layers of air and soft tissue, is more different from bone than other soft tissues such as muscle. Further, Sorenson teaches that Snell's law typically causes most transmitted energy to be reflected along a line which is at an angle to a longitudinal axis of the transmitting transducer. Therefore, Sorenson teaches extensive amplification of the small amount of reflected energy returning along this axis or, in the alternative, capture of reflected energy at one or more additional transducers. Thus, Sorenson teaches determination of co-ordinates of a point in three degrees of freedom, as opposed to six degrees of freedom. Thus changes in an angle of a surface over distance are not determined by these teachings. This is a distinct and inherent disadvantage which renders these teachings unsuitable to use in imaging long bones.

U.S. Pat. No. 5,140,988 issued to Stouffer et al. is a method and apparatus for imaging bone structures in animal carcasses. FIGS. 2 and 3 of this patent demonstrate that the teachings of Stouffer relate to 2 dimensional images of bone. Thus, it is an inherent disadvantage of Stouffer that three dimensional images of bone are not provided.

U.S. Pat. No. 5,840,029 issued to Mazess et al is a method for using ultrasound to measure bone. Mazess concers himself primareily with measurement of bone properties. Mazess contains neither a hint nor suggestion that it is feasible or desireable to generate a 3D image of a bone.

U.S. Pat. No. 5,879,301 issued to Chibrera et al. is a method for detecting the properties of bone using ultrasound, specifically for detecting osteoporosis. It is an inherent disadvantage of Chibrera that production of images of measured bones is not taught.

U.S. Pat. No. 6,015,383 issued to Buhler et al. teaqches acoustic analysis0 to detect the characteristics of bone tissue where the edge of the bone is detected. However, FIGS. 3-6 of this patent make it abundantly clear that while data may be collected in three dimensions, output is supplied as graphs. Thus, it is an inherent disadvantage of Buhler that images are not provided as a result of the scan. It follows that three dimensional images of bone are not provided.

U.S. Pat. No. 322,507 issued to Passi et al. is an ultrasonic system for evaluation bone tissue. Like other patents in this group, it has the inherent disadvantage of providing output as graphs rather than images. Further, measurements according to these teachings are of acoustic properties and not of surfcae position co-ordinates.

Thus, while members of this second group of patents teach assays of bone using ultrasound technology, they fail to teach production of a three dimensional image of a long bone.

Additional patents dealing with ultrasonic imaging of bone are summarized hereinbelow.

U.S. Pat. No. 5,305,752 issued to Spivey is a system for imaging tissue in the body using acoustic waves. While Spivey teaches formation of a single ultrasonic image depicting both soft tissue and bone, the image is a cross-sectional image (i.e. 2 dimensional). Further, independent claim 1 of Spivey teaches dispersing a plurality of closely spaced acoustic signal detection means around a sample to be imaged. This is an additional distinct and inherent disadvantage of the teachings of Spivey.

U.S. Pat. No. 5,465,722 issued to Fort et al. relates to a 3D ultrasonic system. Although these teachings included production of a 3D image of a bone (FIG. 13), production of this image requires receiving reflected acoustic energy at a plurality of locations as optionally taught by Sorenson. Further, Fort teaches transmitting energy from a first set of locations and receiving energy at a second set of locations. This teaching specifically excludes use of specular reflection. These teachings are difficult to implement because of the complexity in processing the received signal and differentiating between soft tissue and hard tissue reflection.

U.S. Pat. No. 6,375,616 issued to Soferman et al. is a method for determining fetal weight in utero. Although Soferman teaches application of grey level threshold in order to isolate bones from other tissue in an image, his teachings do not include use of specular reflection to form the image. In contrast, these teachings seem to include gatering of reflected energy from a single transmission at a plurality of points (column 10 lines 1-30). Reference to “E. J. Feleppa et al., Two Dimensional and Three-Dimensional Tissue-Type Imaging of the Prostate Based on Ultrasonic Spectrum Analysis and Neural-Network Classification”. Suggests that Soferman is aware of the fact that fetal bone, because it is not a specular reflector, is actually more similar to soft tissue than to fully calcified bone. Therefore, direct application of the teachings of. This is a distinct and inherent disadvantage which would make transfer of these teachings to imaging of adult bone problematic.

U.S. Pat. No. 6,390,982 issued to Bova et al. is a method of creating a three dimensional image. The teachings of Bova are directed to ultrasonic probes as an adjunct to a second imaging technology in localizing bone. This means that generation of the three dimensional image from ultrasound image data alone is beyond the scope of Bova's teachings. This is a distinct and inherent disadvantage. Further these teachings rely upon a probe as taught in U.S. Pat. No. 5,893,832 to Song which relies upon collection of data at a plurality of points dispersed radially about an axis of a beam of transmitted energy.

U.S. Pat. No. 6,413,215 issued to Wu et al. is an ultrasonic system for detecting the wear of artificial joints. The teachings of Wu rely upon scattering of ultrasonic energy as a result of cavitation events in synovial fluid. Further, Wu teaches output of data as particle size information, not particle position. In summary, these teachings have little relevance to the instant application because cavitation events are not expected to occur in typical measurement of hard tissue such as bone.

There is thus a widely recognized need for, and it would be highly advantageous to have, a system and method of 3 dimensional ultrasonic imaging of hard tissue devoid of the above limitations.

### SUMMARY OF THE INVENTION

According to one aspect of the present invention there is provided a method of creating an ultrasonic image of a hard tissue within a target. The method includes: (a) transmitting from at least one ultrasonic transducer at a defined location a focused beam of ultrasonic energy towards the target; (b) adjusting an angle of incidence between the focused beam and a surface of the hard tissue to a normal angle, by positioning the at least one ultrasonic transducer; (c) receiving a significant portion of the energy as an echo-reflection at the defined location; (d) defining the location of the transducer in six degrees of freedom; (e) calculating a set of position co-ordinates for a portion of a surface causing the echo-reflection; (f) moving the ultrasonic transducer to a different defined location; (g) repeating steps a through f; and (h) compiling at least a portion of the sets of position co-ordinates to generate a map of at least a portion of the surface causing the echo-reflection; (i) determining at least a portion of the map which represents a surface of the hard tissue within the target according to a predetermined rule.

According to another aspect of the present invention there is provided a system for creating an ultrasonic image of a hard tissue within a target. The system includes: (a) at least one ultrasonic transducer positioned at a defined location and capable of transmitting a focused beam of ultrasonic energy towards the target and of receiving a significant portion of the energy as an echo-reflection from a surface of the hard tissue and of communication with a central processing unit; (b) a position locator and adjustment mechanism coupled to the at least one transducer and designed and constructed to be capable of adjusting an angle of incidence between the focused beam and the surface of the hard tissue in response to a command from the central processing unit and to be capable of defining the location of the transducer in six degrees of freedom and transmitting the definition to the central processing unit as well as to be capable of moving the ultrasonic transducer to a series of different defined location; (c) the central processing unit designed and configured to transmit commands to the position locator and adjustment mechanism to cause the transducer to move to the series of different defined locations, to calculate a set of position co-ordinates for at least a portion of the surface of the hard tissue causing the echo-reflection and to compile a plurality of the sets of position co-ordinates to generate a map of at least a portion of the surface of the hard tissue by applying a predetermined rule.

According to yet another aspect of the present invention there is provided a method of creating an ultrasonic image of a hard tissue including irregularities thereupon, the method includes:

(a) transmitting a focused beam of ultrasonic energy from at least one ultrasonic transducer at a first defined location towards a surface of the hard tissue; (b) receiving a portion of the energy as an echo-reflection at at least one second defined location; (c) calculating a set of position co-ordinates corresponding to an ultrasonic reflector for each of the at least one second defined location; (d) repeating steps a through c; (e) deciding if the reflector is a hard tissue according to a first predetermined criteria; (f) deciding if the reflector is an irregularity on the surface of hard tissue according to a second predetermined criteria; and (g) compiling at least a portion of the sets of position co-ordinates to generate a map of at least a portion of the surface of the hard tissue.

According to still another aspect of the present invention there is provided a system for creating an ultrasonic image of a hard tissue and any irregularities thereupon within a target. The system includes: (a) at least one ultrasonic transmitter capable of transmitting a focused beam of ultrasonic energy from at least one first defined location towards a surface of the hard tissue and further capable of communication with a central processing unit; (b) at least one ultrasonic receiver capable of receiving a portion of the energy as an echo-reflection at at least one second defined location and further capable of communication with the central processing unit; (c) a position locator and adjustment mechanism operably connectable to the at least one transmitter and the at least one receiver and capable of communication with the central processing unit and designed and constructed to be capable of moving the transmitter and the receiver to a series of different defined locations; and (d) the central processing unit. The central processing unit is designed and configured to: (i) calculate a set of position co-ordinates corresponding to an ultrasonic reflector for each of the at least one second defined location; (ii) decide if the reflector is a hard tissue according to a first predetermined criteria; (iii) decide if the reflector constitutes an irregularity on the surface of the hard tissue according to a second predetermined criteria; (iv) compile at least a portion of the sets of position co-ordinates to generate a map of at least a portion of the surface of the hard tissue; and (v) transmit commands to the position locator and adjustment mechanism to cause the transducer to move to the series of different defined locations.

According to further features in preferred embodiments of the invention described below, controlling includes at least one item selected from the group consisting of the adjusting and the moving.

According to still further features in the described preferred embodiments at least one item selected from the group consisting of the adjusting and the moving is performed manually by a practitioner of the method.

According to still further features in the described preferred embodiments performed manually indicates at least one manual input selected from the group consisting of a manual position adjustment by the practitioner of the method and at least one instruction transmitted to the central processing unit by the practitioner of the method.

According to still further features in the described preferred embodiments the angle of incidence is a normal angle determined by moving the at least one ultrasonic transducer.

According to still further features in the described preferred embodiments each of the defined locations is defined as a set of position co-ordinates.

According to still further features in the described preferred embodiments each first defined location includes at least one angle of transmission as part of its definition.

According to still further features in the described preferred embodiments the method further includes employing additional first defined locations for the transmitting. Additional first defined locations include, but are not limited to, transmitting in additional directions (i.e. at different angles) from a single defined location.

According to still further features in the described preferred embodiments the method further includes employing additional second defined locations for the receiving.

According to still further features in the described preferred embodiments the method the controlling includes repositioning at least one item selected from the group consisting of at least one of the at least one ultrasonic transducer and an ultrasonic receiver.

According to still further features in the described preferred embodiments the predetermined rule is selected from a group consisting of a geometric rule and a physical rule. A combination including a geometric rule and/or a physical rule is therefore included.

According to still further features in the described preferred embodiments the predetermined rule includes maximization of the function:
F(x,y,r1.r2,r3)=Σ(ref1(Area 1))−C*Σ(ref2(Area 2))

wherein (x, y) represent an assumed position coordinate within a slice of the hard tissue within the target; and

wherein r1, r2 and r3 each individually represent a radius of the hard tissue with respect to the assumed position co-ordinate at a series of angles α1, α2 and α3 respectively;

wherein ref1 represents a sum of the portion of energy received as an echo reflection within a first area (Area 1) and ref2 represents a sum of the portion of energy received as an echo reflection within a second area (Area 2); and wherein C represents a constant.

According to still further features in the described preferred embodiments the method further includes controlling, by means of a central processing unit, performance of at least a portion of the method.

According to still further features in the described preferred embodiments controlling indicates at least one control mechanism selected from the group consisting of mechanical control, selection from an array and electronic control.

According to still further features in the described preferred embodiments the position locator and adjustment mechanism employs at least one type of control selected from the group consisting of mechanical control, selection from an array and electronic control.

According to still further features in the described preferred embodiments the position locator and adjustment mechanism is further designed and configured to receive input from an operator of the system, the input being selected from the group consisting of a manual position adjustment by an operator of the system and at least one instruction transmitted to the central processing unit.

According to still further features in the described preferred embodiments at least one item selected from the group consisting of data pertaining to the echo-reflection, the set of position co-ordinates for a portion of the surface of the hard tissue causing the echo-reflection and at least a portion of the map is displayed upon a display device.

According to still further features in the described preferred embodiments the map is selected from the group consisting of a two dimensional map and a three dimensional map.

The present invention successfully addresses the shortcomings of the presently known configurations by providing systems and methods of 3 dimensional ultrasonic imaging of hard tissue and/or imperfections contained therein (e.g. fractures, jount abnormalities and implanted surgical anchors.) Preferably, images produced by the system additionally contain depictions of soft tissue surrounding the bone, as part of an integrated 3D rendering.

Implementation of the method and system of the present invention involves performing or completing selected tasks or steps manually, automatically, or a combination thereof. Moreover, according to actual instrumentation and equipment of preferred embodiments of the method and system of the present invention, several selected steps could be implemented by hardware or by software on any operating system of any firmware or a combination thereof. For example, as hardware, selected steps of the invention could be implemented as a chip or a circuit. As software, selected steps of the invention could be implemented as a plurality of software instructions being executed by a computer using any suitable operating system. In any case, selected steps of the method and system of the invention could be described as being performed by a data processor, such as a computing platform for executing a plurality of instructions.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a simplified flow diagram illustrating a sequence of events involved in performance of a method according to the present invention.

FIG. 2 is a schematic representation of components of a system according to the present invention.

FIG. 3 is a simplified flow diagram illustrating a sequence of events involved in performance of an additional method according to the present invention.

FIG. 4 is a schematic representation of components of an additional system according to the present invention.

FIGS. 5 a-c illustrate possible arrangements of ultrasonic transducers with respect to a target according to the present invention.

FIGS. 6 a and b present maps produced by the present invention.

FIG. 7 is a diagram illustrating the physical relationship among variables in a formula presented in support of the present invention.

### DESCRIPTION OF THE PREFERRED EMBODIMENTS

The present invention is of systems and methods of ultrasonic imaging of hard tissue which can be used to produce 3 dimensional maps or images of hard tissue surfaces and/or irregularities thereupon.

Specifically, the present invention can be used to image imperfections in bone (e.g. fractures, jount abnormalities and implanted surgical anchors.) Preferably, images produced by the present invention additionally contain depictions of soft tissue surrounding the bone, as part of an integrated 3D rendering.

The principles and operation of systems and methods according to the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

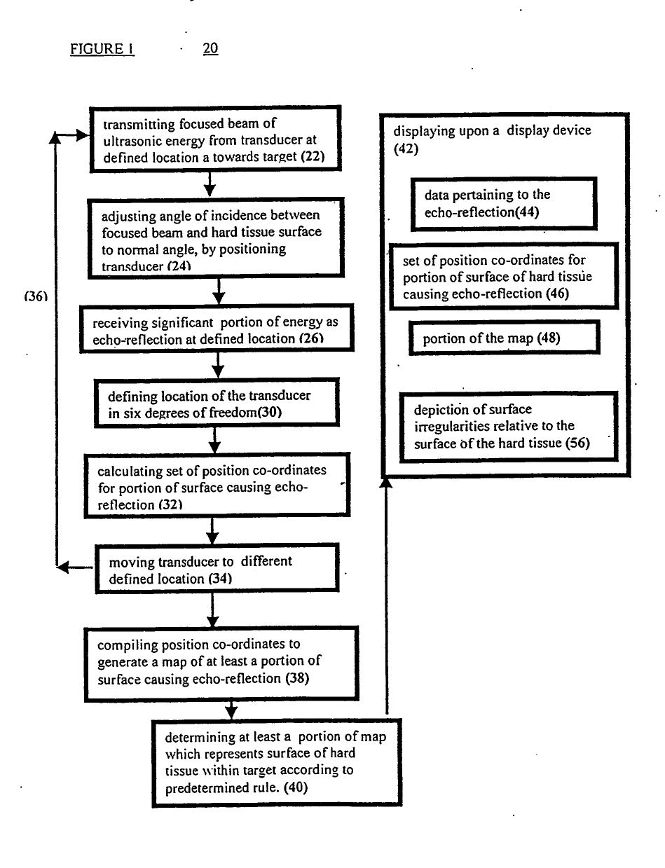

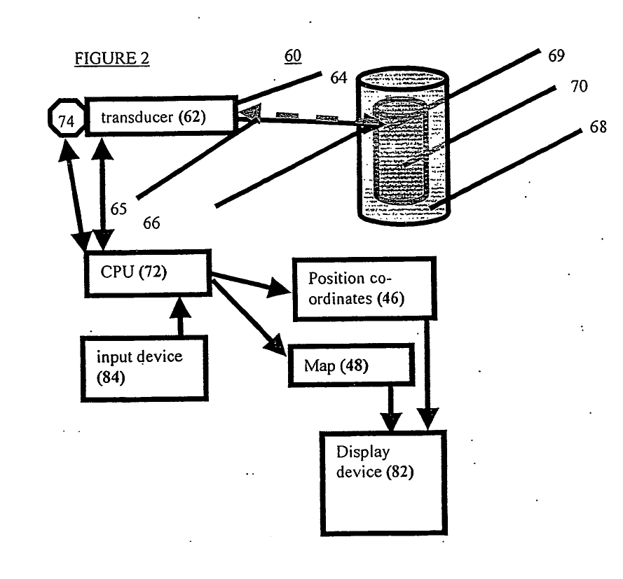

The present invention is embodied by a method 20 (FIG. 1) of creating an ultrasonic image 48 of a hard tissue 70 (FIG. 2) within a target 68.

For purposes of this specification and the accompanying claims, the term “ultrasonic” pertains to sound waves with a frequency in excess of approximatelt 20 kHz, more preferably in the range of 1 mHz to 20 mHz.

For purposes of this specification and the accompanying claims, the term “hard tissue” includes, but is not limited to bone such as cortical bone or trebicular bone. Bone refers to calcified fully developed bone capable of generating an ultrasonic echo-reflection in approximate accordance with Snell's law. While a developing fetus may have bones, these fetal bones are excluded from the definition of hard tissue because they are primarily cartilaginous, significant calcification typically taking place well after parturition.

The term “target” as used in this specification and the accompanying claims indicates any portion of a subject which may be under examination.

Method 20 includes transmitting 22 from at least one ultrasonic transducer 62 at a defined location 64 a focused beam 66 of ultrasonic energy towards target 68. Preferably, beam 66 is pulsed.

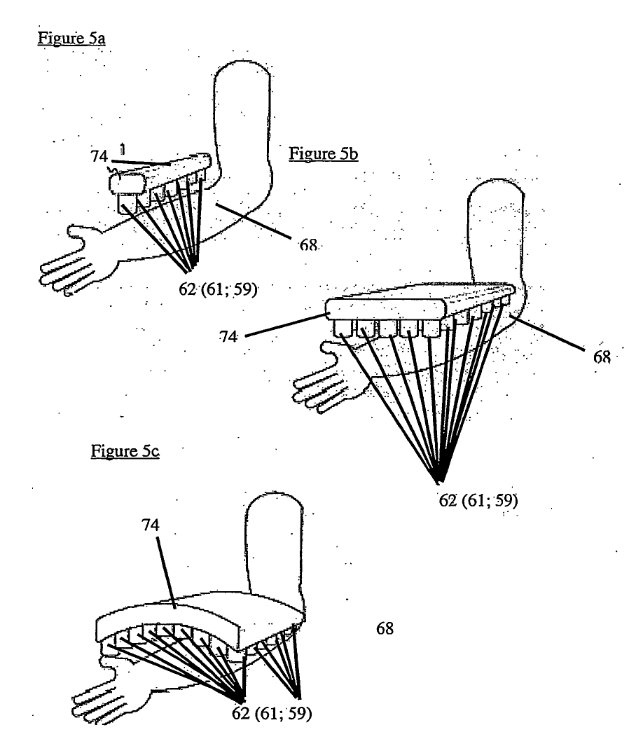

For purposes of this specification and the accompanying claims, the term “focused” means that a majority of transmitted energy is concentrated within a defined area surrounding an axis of a transmitted beam. Focusing may be caused by interference of a plurality of transmitted beams. Depending upon the arrangement of transmitting transducers 62 (see FIGS. 5a-c), this interference may involve a temporal component (i.e. transducers located further from the target transmitting earlier, those located closer to the target transmitting later) as well as a spatial component.

For purposes of this specification and the accompanying claims, the term “beam” indicates a ray of energy transmitted from one or more sources.

For purposes of this specification and the accompanying claims, the term “pulsed” means temporally defined.

Method 20 further includes adjusting 24 an angle of incidence between focused beam 66 and a surface 69 of the hard tissue 70 to a normal angle, by positioning the at least one ultrasonic transducer 62.

For purposes of this specification and the accompanying claims, the term “normal angle” refers to an angle at which a single ultrasonic transducer at a fixed location can transmit energy and receive an echo reflection of a significant portion of the transmitted energy. In theory a normal angle is 0° (zero degrees), and a 0° angle is always optimal. In practice the range of normal angles is a function of surface properties of the hard tissue under examination so that 0°±15°, more preferably 0°±10°, most preferably 0°±5°, is functionally normal for purposes of generating a measurable echo reflection.

Method 20 further includes receiving 26 a significant portion 65 of the energy as an echo-reflection at defined location 64. Method 20 further includes defining 30 location 64 of transducer 62 in six degrees of freedom.

Method 20 further includes calculating 32 a set of position co-ordinates 46 for a portion of a surface causing the echo-reflection. Method 20 further includes moving 34 ultrasonic transducer 62 to a different defined location 64, and repeating 36 transmitting 22, adjusting 24, receiving 26, defining 30, calculating 32 and moving 34. Repeating 36 is preferably performed many times.

Method 20 further includes compiling 38 at least a portion of the sets of position co-ordinates 46 to generate a map 48 of at least a portion of surface 69 causing echo-reflection 65.

Method 20 further includes determining 40 at least a portion of map 48 which represents surface 69 of hard tissue 70 within target 68 according to a predetermined rule.

Optionally, but preferably, a computerized controller 72 is employed to control at leasat a portion method 20, for example adjusting 24 or moving 34. Computerized controller 72 may be, for example, a computer such as a personal computer (PC) having an operating system such as DOS, Windows™, OS/2™ or Linux; Macintosh™, Palm OS™, an EPOC™ computer; a computer having JAVA™-OS as the operating system; a graphical workstations such as a computer of Sun Microsystems™ or Silicon Graphics™, or another computer having some version of the UNIX operating system such as AIX™ or SOLARIS™ of Sun Microsystems™; or any other known and available operating system, or a personal digital assistant (PDA), each of which is known to include an inherent or connectable display device 82.

Optionally, but also preferably, adjusting 24 and/or moving 34 may be performed manually by a practitioner of method 20. Manually, as used herein, indicates at least one manual input. A manual input may be, for example, a manual position adjustment by the practitioner of method 20 or at least one instruction transmitted to the central processing unit 72 by the practitioner of method 20 (e.g. via input device 84). Input device 84 may be any device for entry of data to a computing device. Therefore, input device 84 may include, but is not limited to, a keyboard, a computer mouse, a trackpad, a track ball, a stylus, a touchscreen and a microphone.

Practice of method 20 typically involves use of a system 60 which further embodies the prsent invention. System 60 creates an ultrasonic image of hard tissue 70 within target 68.

System 60 includes at least one ultrasonic transducer 62 positioned at defined location 64 and capable of transmitting 22 focused beam 66 of ultrasonic energy towards target 68 and of receiving 26 significant portion 65 of the energy as an echo-reflection from surface 69 of hard tissue 70 and of communication with central processing unit 72. Preferably the angle of incidence between beam 66 and surface 69 of hard tissue 70 is a normal angle determined by moving transducer 62 so that echo reflection 65 is maximized.

System 60 further includes a position locator and adjustment mechanism 74 coupled to at least one transducer 62 and designed and constructed to be capable of adjusting 24 an angle of incidence between focused beam 66 and surface 69 of hard tissue 70 in response to a command from central processing unit 72. Position locator and adjustment mechanism 74 is further capable of defining 30 location 64 of transducer 62 in six degrees of freedom. Position locator and adjustment mechanism 74 is further capable of transmitting the definition to central processing unit 72. Position locator and adjustment mechanism 74 is further capable of moving ultrasonic transducer 62 to a series of different defined locations 64.

System 60 further includes central processing unit 72 designed and configured to transmit commands to position locator and adjustment mechanism 74 to cause transducer 62 to move to a series of different defined locations 64. Central processing unit 72 is further designed and configured to to calculate position co-ordinates 46 for at least a portion of surface 69 of hard tissue 70 causing echo-reflection 65 and to compile a plurality position co-ordinates 46 to generate map 48 of at least a portion of surface 69 of hard tissue 70 by applying a predetermined rule.

Preferably the predetermined rule employed by CPU 72 in determining 40 map 48 representing surface 69 of hard tissue 70 includes a geometric rule or a physical rule or a combination thereof.

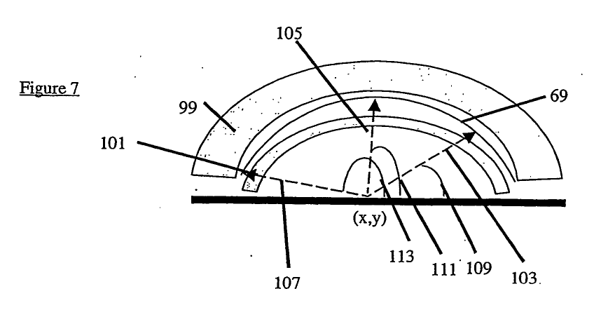

Maximization of the function:
F(x,y,r1.r2,r3)=Σ(ref1(Area 1))−C*Σ(ref2(Area 2)) (see FIG. 7)

is presented as a non limiting example of a predetermined rule suited for use in the context of the present invention

According to this function:

(x, y) represent an assumed position coordinate within a slice of hard tissue 70 within target 68; and

r1, r2 and r3 (103, 105 and 107 respectively) each individually represent a radius of hard tissue 70 with respect to (x, y) at a series of angles α1, α2 and α3 respectively (109, 111 and 113 respectively); and

ref1 represents a sum of the portion of energy received as echo reflection 65 within a first area (Area 1; 99) and ref2 represents a sum of the portion of energy received as echo reflection 65 within a second area (Area 2 101); and

C represents a constant.

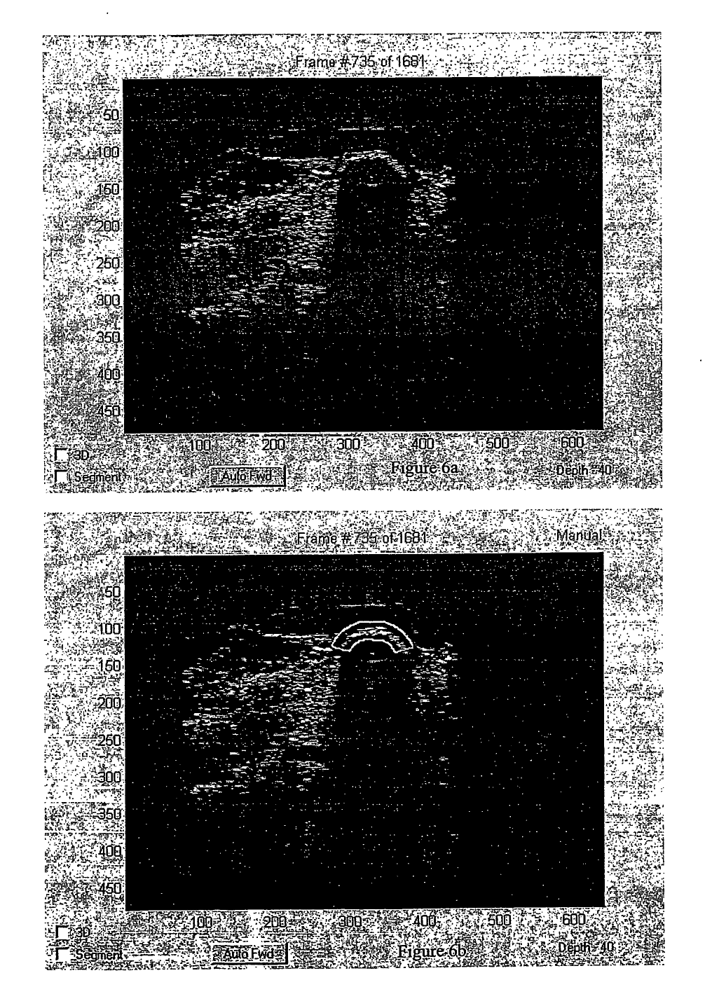

FIGS. 6 a and b are images of surface 69 of hard tissue 70 produced using maximization of this function.

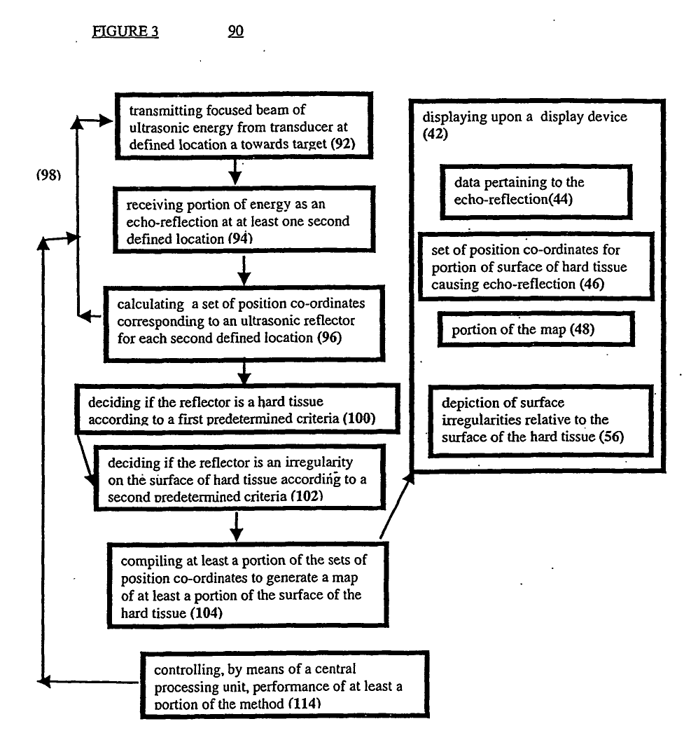

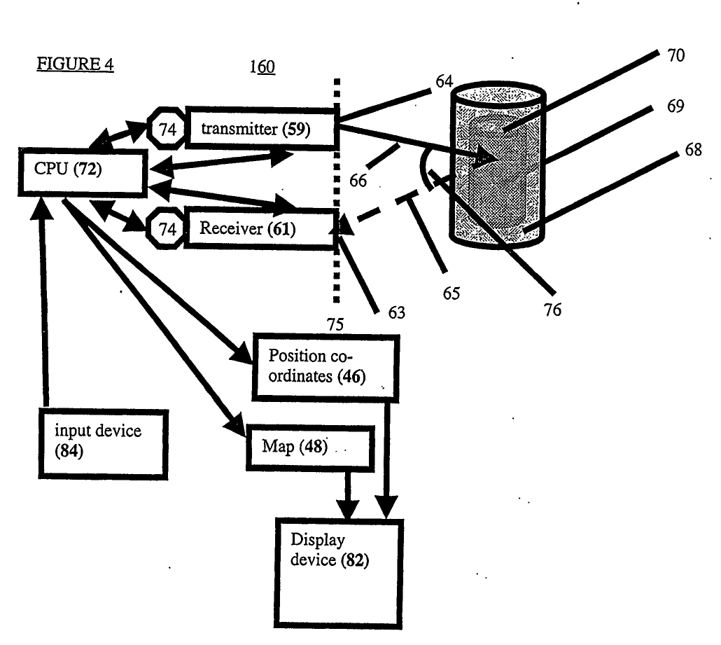

The present invention is further embodied by an additional method 90 of creating an ultrasonic image of a hard tissue including irregularities thereupon. Method 90 includes transmitting 92 (FIG. 3) a focused beam 66 (FIG. 4) of ultrasonic energy from at least one ultrasonic transducer, pictured here as transmitter 59 for clarity, at a first defined location 64 towards a surface 69 of hard tissue 70.

Method 90 further includes receiving 94 a portion 65 of the energy as an echo-reflection at at least one second defined location 63. The nature of surface 69 will determine the number of locations 63 (and receivers 61) which are optimal. In practice, both receiver 61 and transmitter 59 will usually both be transducers 62, although this is not always the case.

Method 90 further includes calculating 96 a set of position co-ordinates 46 corresponding to an ultrasonic reflector for each second defined location 63 at which energy 65 is received.

Method 90 further includes repeating transmitting 92, receiving 94 and calculating 96.

Method 90 further includes deciding 100 if the reflector is a hard tissue according to a first predetermined criteria and deciding 102 if the reflector is an irregularity on the surface of hard tissue according to a second predetermined criteria.

The first predetermined criteria may be, for example, definition of a small area upon which echo reflection 65 is distributed. Confineminemt of reflection 65 to defined small area indicates that it is caused by a hard surface.

The second predetermined criteria may be, for example, definition of a larger area upon which echo reflection 65 is distributed. Dispersal of reflection 65 to a larger area indicates that it is caused by an irregularity.

Alternately, or additionally, first and second predetermined criteria may include analysis of the geometry of the area in which reflection 65 is received by geometrical properties of the area

Method 90 further includes compiling 104 at least a portion of the sets of position co-ordinates 46 to generate map 48 of at least a portion of surface 69 of hard tissue 70.

Thus, determination of a position co-ordinate 46 is preferably accomplished by comparing received energy 65 at position 63 with transmitted energy 66. In order to make this determination, the positions of transmission origin 64 and receiver position 63 relative to one another must be known. Alternately, but also preferably, determination of a position co-ordinate 46 is accomplished by analyzing the pattern of energy 65 received 94 at many. Thus it is often useful to employ arrays of transducers 62 in systems 60 or 160 (FIG. 4) as pictured in FIGS. 5 a-c in order to quickly gather data required for performance of methods 90 and/or 20.

Performance of methods 90 and/or 20 is most efficient when each of defined locations 64 and 63 is defined as a set of position co-ordinates. Preferably, each first defined location 64 includes at least one angle of transmission as part of its definition.

Preferably method (20 or 90) further includes employing additional first defined locations 64 for transmitting (22; 92). Additional first defined locations 64 include, but are not limited to, transmitting in additional directions (i.e. at different angles) from a single defined location 64. Preferably method 90 further includes employing additional second defined locations 93 for receiving 94. Arrays of transducers 62 as shown in FIGS. 5a through c are useful in this respect as they increase the speed at which position co-ordinates 46 may be generated by increasing the speed at which transmission/reception from a large number of locations may be accomplished.

In association with methods 20 and 90. controlling may include repositioning transducer 62, transmitter 59 or ultrasonic receiver 61, whether by means of choosing an additional item from an array or by physically moving an item.

Methods 20 and/or 90 are both amenable to controlling, by means of a central processing unit, performance of at least a portion of the method. This automated control enhances the speed and performance of 20 and/or 90. Controlling may indicate, for example, use of a control mechanism such as mechanical controller, selection from an array, electronic control or combinations including any of same.

System 160 for creating an ultrasonic image of a hard tissue and any irregularities thereupon within a target is depicted in FIG. 4. System 160 includes at least one ultrasonic transmitter 59 capable of transmitting 92 beam 66 from at defined location 64 towards surface 69 of hard tissue 70 and further capable of communication with central processing unit 72.

System 160 further includes ultrasonic receiver 61 capable of receiving 94 a portion 65 of the energy as an echo-reflection at second defined location 63 and further capable of communication with central processing unit 72.

System 160 further includes a position locator and adjustment mechanism 74 operably connectable to transmitter 59 and receiver 61 and capable of communication with central processing unit 72. Position locator and adjustment mechanism 74 is designed and constructed to be capable of moving transmitter 59 and receiver 61 to a series of different defined locations (64; 63). Position locator and adjustment mechanism 74 may employs, for example, a mechanical control mechanism, selection from an array or an electronic control mechanism.

Position locator and adjustment mechanism 74 is optionally, but preferably, designed and configured to receive input from an operator of the system (60; 160). The input may be, for example, a manual position adjustment by an operator of the system or at least one instruction transmitted to central processing unit 72 (e.g. by data input device 84).

System 160 further includes central processing unit 72 designed and configured to calculate 96 a set of position co-ordinates 46 corresponding to an ultrasonic reflector for each second defined location 63. Central processing unit 72 is further designed and configured to decide 100 if the reflector is a hard tissue according to a first predetermined criteria. Central processing unit 72 is further designed and configured to decide 102 if the reflector constitutes an irregularity on the surface of the hard tissue according to a second predetermined criteria. Central processing unit 72 is further designed and configured to compile 104 at least a portion of the sets of position co-ordinates 46 to generate a map 48 of at least a portion of the surface of the hard tissue. Central processing unit 72 is further designed and configured to transmit commands (e.g. control 114) to the position locator and adjustment mechanism to cause the transducers (59; 61) to move to different defined locations (64; 63).

Preferably transmitting 92 and receiving 94 occur in a single plane 75 and transmitting 92 of beam 66 is at a normal angle with repect to plane 75.

Practice of methods 20 and 90 typically includes display 42 of output on display device 82. Display device 82 may include any device which visually presents data to a user. Therefore, display device 82 may be, for example, a cathode ray tube display screen, a liquid crystal display, a print out, a plasma screen or an array of light emitting diodes. Similarly, systems 60 and 160 preferably include such display device 82. Displayed output may include, but is not limited to data 44 pertaining to echo-reflection 65, position co-ordinates 46 for a portion of surface 69 of hard tissue 70 causing echo-reflection 65; and at least a portion of map 48 (e.g. FIGS. 6a and 6b). Preferably, surface 69 of hard tissue 70 is visually highlighted (FIG. 6b). Map 48 may be presented as a two dimensional map or a three dimensional map. Preferably surface irregularities 56 are also displayed. More preferably, soft tissue of target 68 is further displayed.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A method of creating an ultrasonic image of a hard tissue within a target, the method comprising:
(a) transmitting from at least one ultrasonic transducer at a defined location a focused beam of ultrasonic energy towards the target;
(b) adjusting an angle of incidence between said focused beam and a surface of the hard tissue to a normal angle, by positioning said at least one ultrasonic transducer;
(c) receiving a significant portion of said energy as an echo-reflection at said defined location;
(d) defining said location of said transducer in six degrees of freedom;
(e) calculating a set of position co-ordinates for a portion of a surface causing said echo-reflection;
(f) moving said ultrasonic transducer to a different defined location;
(g) repeating steps a through f; and
(h) compiling at least a portion of said sets of position co-ordinates to generate a map of at least a portion of said surface causing said echo-reflection;
(i) determining at least a portion of said map which represents a surface of the hard tissue within the target according to a predetermined rule.

2. The method of claim 1, wherein said predetermined rule is selected from a group consisting of a geometric rule and a physical rule.

3. The method of claim 1, wherein said predetermined rule includes maximization of the function:
F(x,y,r1.r2, r3)=Σ(ref1(Area 1))−C*Σ(ref2(Area 2))
wherein (x, y) represent an assumed position coordinate within a slice of the hard tissue within the target; and
wherein r1, r2 and r3 each individually represent a radius of the hard tissue with respect to said assumed position co-ordinate at a series of angles α1, α2 and α3 respectively;
wherein ref1 represents a sum of said portion of energy received as an echo reflection within a first area (Area 1) and ref2 represents a sum of said portion of energy received as an echo reflection within a second area (Area 2); and wherein C represents a constant.

4. The method of claim 1, further comprising:
(i) displaying upon a display device at least one item selected from the group consisting of:
(i) data pertaining to said echo-reflection;
(ii) said set of position co-ordinates for a portion of said surface of the hard tissue causing said echo-reflection; and
(iii) said map.

5. The method of claim 1, further comprising:
(i) controlling, by means of a central processing unit, performance of at least a portion of the method.

6. The method of claim 5, wherein said controlling includes at least one item selected from the group consisting of said adjusting and said moving.

7. The method of claim 6, wherein said controlling indicates at least one control mechanism selected from the group consisting of mechanical control, selection from an array and electronic control.

8. The method of claim 1, wherein at least one item selected from the group consisting of said adjusting and said moving is performed manually by a practitioner of the method.

9. The method of claim 8, wherein said performed manually indicates at least one manual input selected from the group consisting of a manual position adjustment by said practitioner of the method and at least one instruction transmitted to said central processing unit by said practitioner of the method.

10. The method of claim 1, wherein said map is selected from the group consisting of a two dimensional map and a three dimensional map.

11. A system for creating an ultrasonic image of a hard tissue within a target, the system comprising:
(a) at least one ultrasonic transducer:
(i) said at least one transducer positioned at a defined location;
(ii) said at least one transducer capable of transmitting a focused beam of ultrasonic energy towards the target;
(iii) said at least one transducer capable of receiving a significant portion of said energy as an echo-reflection from a surface of the hard tissue; and
(iv) said at least one transducer transducer capable of communication with a central processing unit;
(b) a position locator and adjustment mechanism coupled to said at least one transducer;
(i) said position locator and adjustment mechanism designed and constructed to be capable of adjusting an angle of incidence between said focused beam and said surface of the hard tissue in response to a command from said central processing unit;
(ii) said position locator and adjustment mechanism further designed and constructed to be capable of defining said location of said transducer in six degrees of freedom and transmitting said definition to said central processing unit;
(iii) said position locator and adjustment mechanism further designed and constructed to be capable of moving said ultrasonic transducer to a series of different defined location;
(c) said central processing unit designed and configured to;
(i) transmit commands to said position locator and adjustment mechanism to cause said transducer to move to said series of different defined locations
(ii) calculate a set of position co-ordinates for at least portion of said surface of the hard tissue causing said echo-reflection;
(iii) compile a plurality of said sets of position co-ordinates to generate a map of at least a portion of said surface of the hard tissue by applying a predetermined rule.

12. The system of claim 11, further comprising a:
(d) a display device capable of communication with said central processor; said display device designed and constructed to perform at least one function selected from the group consisting of:
(i) display data pertaining to said echo-reflection;
(ii) display said set of position co-ordinates for a portion of said surface of the hard tissue causing said echo-reflection; and
(iii) display at least a portion of said map.

13. The system of claim 11, wherein said angle of incidence is a normal angle determined by moving said at least one ultrasonic transducer.

14. The system of claim 11, wherein said position locator and adjustment mechanism employs at least one type of control selected from the group consisting of mechanical control, selection from an array and electronic control.

15. The system of claim 11, wherein said position locator and adjustment mechanism is further designed and configured to receive input from an operator of the system, said input being selected from the group consisting of a manual position adjustment by an operator of the system and at least one instruction transmitted to said central processing unit.

16. The system of claim 11, wherein said map is selected from the group consisting of a two dimensional map and a three dimensional map.

17. The system of claim 11, wherein said predetermined rule is selected from a group consisting of a geometric rule and a physical rule.

18. The system of claim 11, wherein said predetermined rule includes maximization of the function:
F(x,y,r1.r2,r3)=Σ(ref1(Area 1))−C*Σ(ref2(Area 2))
wherein (x, y) represent an assumed position coordinate within a slice of the hard tissue within the target; and
wherein r1, r2 and r3 each individually represent a radius of the hard tissue with respect to said assumed position co-ordinate at a series of angles α1, α2 and α3 respectively;
wherein ref1 represents a sum of said portion of energy received as an echo reflection within a first area (Area 1) and ref2 represents a sum of said portion of energy received as an echo reflection within a second area (Area 2); and wherein C represents a constant.

19. A method of creating an ultrasonic image of a hard tissue including irregularities thereupon, the method comprising:
(a) transmitting a focused beam of ultrasonic energy from at least one ultrasonic transducer at a first defined location towards a surface of the hard tissue;
(b) receiving a portion of said energy as an echo-reflection at at least one second defined location;
(c) calculating a set of position co-ordinates corresponding to an ultrasonic reflector for each of said at least one second defined location;
(d) repeating steps a through c;
(e) deciding if said reflector is a hard tissue according to a first predetermined criteria;
(f) deciding if said reflector is an irregularity on the surface of hard tissue according to a second predetermined criteria; and
(g) compiling at least a portion of said sets of position co-ordinates to generate a map of at least a portion of said surface of the hard tissue.

20. The method of claim 19, wherein each of said defined locations is defined as a set of position co-ordinates.

21. The method of claim 19, wherein said first defined location include angles of transmission.

22. The method of claim 19, further including employing additional first defined locations for said transmitting.

23. The method of claim 19, further including employing additional second defined locations for said receiving.

24. The method of claim 19, further comprising:
(h) displaying upon a display device at least one item selected from the group consisting of:
(i) data pertaining to said echo-reflection;
(ii) said set of position co-ordinates for said portion of the surface of the hard tissue causing said echo-reflection; and
(iii) at least a portion of said map.

25. The method of claim 19, further comprising:
(h) controlling, by means of a central processing unit, performance of at least a portion of the method.

26. The method of claim 25, wherein said controlling includes repositioning at least one item selected from the group consisting of at least one of said at least one ultrasonic transducer and an ultrasonic receiver.

27. The method of claim 25, wherein said controlling indicates at least one control mechanism selected from the group consisting of mechanical control, selection from an array and electronic control.

28. The method of claim 19, wherein said map is selected from the group consisting of a two dimensional map and a three dimensional map.

29. A system for creating an ultrasonic image of a hard tissue and any irregularities thereupon within a target, the system comprising:
(a) at least one ultrasonic transmitter capable of transmitting a focused beam of ultrasonic energy from at least one first defined location towards a surface of the hard tissue and further capable of communication with a central processing unit;
(b) at least one ultrasonic receiver capable of receiving a portion of said energy as an echo-reflection at at least one second defined location and further capable of communication with said central processing unit;
(c) a position locator and adjustment mechanism operably connectable to said at least one transmitter and said at least one receiver and capable of communication with said central processing unit and designed and constructed to be capable of moving said transmitter and said receiver to a series of different defined locations; and
(d) said central processing unit designed and configured to:
(i) calculate a set of position co-ordinates corresponding to an ultrasonic reflector for each of said at least one second defined location;
(ii) decide if said reflector is a hard tissue according to a first predetermined criteria;
(iii) decide if said reflector constitutes an irregularity on the surface of the hard tissue according to a second predetermined criteria
(iv) compile at least a portion of said sets of position co-ordinates to generate a map of at least a portion of said surface of the hard tissue; and
(v) transmit commands to said position locator and adjustment mechanism to cause said transducer to move to said series of different defined locations

30. The system of claim 29, further comprising a:
(d) a display device capable of communication with said central processor; said display device designed and constructed to perform at least one function selected from a group consisting of:
(i) display data pertaining to said echo-reflection;
(ii) display said set of position co-ordinates for a portion of said surface of the hard tissue causing said echo-reflection; and
(iii) display at least a portion of said map.

31. The system of claim 29, wherein said position locator and adjustment mechanism employs at least one type of control selected from the group consisting of mechanical control, selection from an array and electronic control.

32. The system of claim 29, wherein said position locator and adjustment mechanism is further designed and configured to receive input from an operator of the system, said input being selected from the group consisting of a manual position adjustment by an operator of the system and at least one instruction transmitted to said central processing unit.

33. The system of claim 29, wherein said map is selected from the group consisting of a two dimensional map and a three dimensional map.
